(19) United States
(12) Patent Application Publication
(10) Pub. No.: US 2023/0364166 A1
(43) Pub. Date: Nov. 16, 2023

(54) APPLICATION OF POSTBIOTICS OF INACTIVATED LACTOBACILLUS CASEI IOB-P9 IN BLOOD GLUCOSE REDUCING

(71) Applicant: Tianjin InnoOrigin Biological Technology Co., Ltd., Tianjin (CN)

(72) Inventors: Xuemei Han, Tianjin (CN); Wu Liang, Tianjin (CN); Haikuang Wang, Tianjin (CN)

(21) Appl. No.: 18/193,655

(22) Filed: Mar. 31, 2023

(30) Foreign Application Priority Data
May 16, 2022 (CN) 202210525330.7

(51) Int. Cl. A61K 35/747; A61P 3/10

(52) U.S. Cl. CPC A61K 35/747; A61P 3/10

(57) ABSTRACT

Application of postbiotics of inactivated Lactobacillus casei I0B-P 9 in blood glucose reducing is provided in this disclosure. Application of inactivated I0B-P9 fermented postbiotics to preparation of article for relieving diabetes is provided, in which Lactobacillus casei I0B-P9 is preserved in China General Microbiological Culture Collection Center, with a preservation number of CGMCC No. 24195. Lactobacillus casei I0B-P9 strain obtained in the disclosure has high activity, and postbiotics of Lactobacillus casei I0B-P9 obtained in the disclosure can effectively delay and control occurrence and development of type II diabetes, and can be used for preparing products for diabetics.

### CROSS REFERENCE TO RELATED APPLICATIONS

This application claims foreign priority of Chinese Patent Application No. 202210525330.7, filed on May 16, 2022 in the China National Intellectual Property Administration, the disclosures of all of which are hereby incorporated by reference.

### TECHNICAL FIELD

The disclosure relates to the technical field of microorganisms, in particular to application of inactivated Lactobacillus casei I0B-P9 and postbiotics thereof in blood glucose reducing.

### BACKGROUND ART

Diabetes is one of the most important non-infectious diseases (NCD) that threaten global human health. According to statistics of the International Diabetes Federation (IDF), the number of diabetic patients in the world has reached 370 million in 2011, of which 80% are in developing countries, and it is estimated that there will be nearly 550 million diabetic patients in the world by 2030. In 2011, a total of 4.6 million people died of diabetes in the world, and global medical expenditure of diabetes in that year reached 465 billion US dollars.

Diabetes is a lifelong disease, which cannot be eradicated and cured at a current medical level, and requires lifelong medication and treatment. There is often no obvious symptoms for early diabetes which are not easy to be detected by patients, so the early diabetes is mainly treated with lifestyle intervention. Living with illness is current situation of all people with diabetes, and thus it is urgent to find a product that can relieve diabetes and delay occurrence of complications. It is particularly important to research a method of treating diabetes instead of conventional drugs.

At present, some functional studies on Lactobacillus casei mainly involve following aspects: Lactobacillus casei strain and its application in Chinese patent application NO. CN201910259094.7, mainly used for antibacterial and anti-inflammatory; a new type of Lactobacillus casei and its application in Chinese patent application NO. CN201610809341.2, which mainly has antibacterial, antioxidant, gastrointestinal health improving and immunity enhancing effects; a compound probiotic bacteria for improving diabetes, including Lactobacillus casei, disclosed in Chinese patent application NO. CN202010135872.4; and Lactobacillus casei for prevention and adjuvant treatment of type II diabetes disclosed in Chinese patent application NO. CN202010424363.3. There is no report on application of inactivated Lactobacillus casei fermented postbiotics in blood glucose reducing. Application of inactivated Lactobacillus casei I0B-P9 and postbiotics thereof in blood glucose reducing are provided in this disclosure, which has important research significance on effect on diabetes and facilitates developing of products that can relieve diabetes symptoms without damaging health and provides ideas for it.

### SUMMARY

Inactivated Lactobacillus casei I0B-P9, a manufacturing process of postbiotics thereof, and application of the inactivated Lactobacillus casei I0B-P9 and the postbiotics thereof in blood glucose reducing and improving of dyslipidemia of diabetics are provided in this disclosure.

The Lactobacillus casei I0B-P9 adopted in the disclosure is obtained by self-screening, and such a strain has been preserved and physical and chemical indexes thereof have been measured. A strain of the Lactobacillus casei I0B-P9 has been preserved in China General Microbiological Culture Collection Center (CGMCC) with an address of No. 3, Yard 1, Beichen West Road, Chaoyang District, Beijing, on Dec. 27, 2021, with a preservation number of CGMCC No. 24195, and classification named Lactobacillus casei.

In order to solve above problems, technical schemes of the disclosure are as follows.

A strain adopted in the disclosure is the Lactobacillus casei I0B-P9. The strain is subjected to bacterial identification and testing, and is with a white, circular, surface wetted, opaque and clean-edged colony.

An activation medium adopted in the disclosure is 1% to 3% of soy powder.

A solid fermentation medium adopted in the disclosure is soy powder with a ratio of material to water of 1:1 to 1:2.

The Lactobacillus casei I0B-P9 was taken out of a cryotube and inoculated into the activation medium, with an inoculation ratio was 10⁶ to 10⁷ CFU/mL, and a condition of strain activation was as follows: a culture temperature of 37±2° C., a culture duration of 20±2 h, and closed culture. The activated strain was a seed liquid.

The activated seed liquid was inoculated into the solid fermentation medium, with an inoculation amount of 1% to 5% of a dry weight of the solid medium, and after being cultured at 37±2° C. for 24 to 48±2 h, the cultured fermented product was dried at a high temperature, with a drying temperature of 55±2° C. and drying moisture content being controlled to be less than or equal to 10%, so as to obtain inactivated I0B-P9 fermented postbiotics.

### Beneficial Effects

The postbiotics of the Lactobacillus casei I0B-P9 according to the disclosure can effectively delay and control type II diabetes, and can be used as a daily product for diabetics.

### BRIEF DESCRIPTION OF THE DRAWINGS

FIG. 1 shows a seed growth curve;

FIG. 2 shows changes in blood glucose levels for different groups;

FIG. 3 shows changes in glucose tolerance for different groups;

FIG. 4 shows liver glycogen contents for different groups of mice;

FIG. 5 shows changes in TC, HDL-c, LDL-c and TG for different groups of mice.

A strain of the Lactobacillus casei I0B-P9 has been preserved in China General Microbiological Culture Collection Center (CGMCC) with an address of No. 3, Yard 1, Beichen West Road, Chaoyang District, Beijing, on Dec. 27, 2021, with a preservation number of CGMCC No. 24195, and classification named Lactobacillus casei.

### DETAILED DESCRIPTION

Embodiment 1 Preparation of postbiotics of Inactivated Lactobacillus Casei I0B-P9

1 Determination of Seed Age

The strain in a cryotube was inoculated into an agarslantculture-medium, which was placed at 37±2° C. for culture, and colony morphology and thallus characteristics at different culture durations were observed.

The agarslantculture-medium is a MRS culture medium, with a formula of: 10.0 g of peptone, 5.0 g of beef extract, 4.0 g of yeast extract, 20.0 g of glucose, 1.0 mL of tween 80, 2.0 g of dipotassium hydrogen phosphate (7H₂O), 5.0 g of sodium acetates (3H₂O), 2.0 g of ammonium citrate tribasic, 0.2 g of magnesium sulfate (7H₂O) and 0.05 g of manganese sulfate (4H₂O), 15.0 g of agar, and with a pH of 6.2±0.2 and with 1 L of water added.

TABLE 1
growth of seeds for different culture duration

| Culture duration | Thallus morphology |
|---|---|
| 14 | less obvious lawns, and rod |
| | like and uniform colony and |
| | small individuals in microscopic |
| | examination |
| 18 | thin and obvious lawns, rod |
| | like and uniform thallus in |
| | microscopic examination |
| 20 | uniform lawns, white and |
| | circular colony, and rod like |
| | thallus and uniform individuals |
| | in microscopic examination |
| 22 | uniform lawns, white, circular, |
| | surface wetted and opaque |
| | colony, and rod like thallus and |
| | uniform individuals in |
| | microscopic examination |
| 24 | uniform lawns, white, circular, |
| | surface wetted, opaque and |
| | clean-edged colony, and rod like |
| | thallus and uniform and robust |
| | individuals in microscopic |
| | examination |
| 26 | uniform lawns, white, circular, |
| | surface wetted, opaque and |
| | clean-edged colony, and rod like |
| | thallus and uniform and robust |
| | individuals in microscopic |
| | examination |
| 28 | uniform lawns, white, circular, |
| | surface wetted, opaque and |
| | clean-edged colony, and rod like |
| | thallus and uniform and robust |
| | individuals in microscopic |
| | examination |

In cultivation, it is required that agar slant seeds are with uniform individuals and consistent morphology and are robust. The slant seeds with culture duration of 24 h are suitable, so optimal culture duration of the slant seeds is determined to be 24 h.

2 Determination of Seed Age for Activation Medium

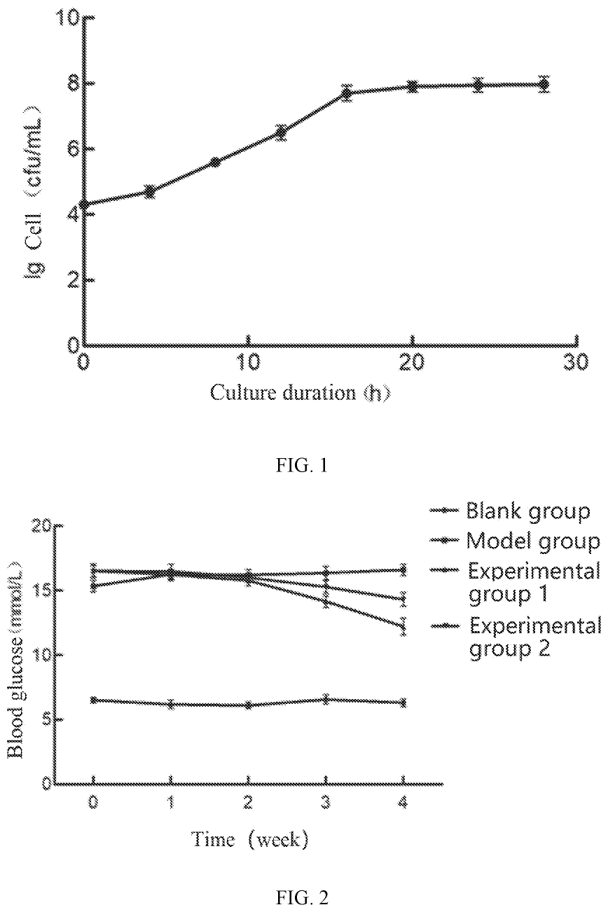

A loop of fresh agar slant strains were inoculated into a bottle filled with 25 mL of 2% soy powder at 37±2° C., and were subjected to closed static culture. Samples with different culture durations were taken for microscopic examination, viable concentration was determined. Results are shown in FIG. 1.

TABLE 2
microscopic examination results of seeds for different duration

| Culture duration | Thallus morphology and number |
|---|---|
| 4 | small amount of thallus |
| 8 | small amount of thallus |
| 12 | short-to-medium rod thallus, a |
| | medium number of thallus |
| 16 | short-to-medium rod thallus, |
| | with uniform sizes and increased |
| | number of thallus |
| 20 | Short rod thallus, with uniform |
| | morphology, mainly in a form of |
| | single colony, a large number of |
| | thallus |
| 24 | Short rod thallus, with uniform |
| | morphology, in a form of single |
| | colony, a large number of |
| | thallus |

As can be seen from FIG. 1 and Table 2, the culture duration is from 16 h to 20 h for which thallus are in a later stage of a logarithmic growth period, at which time the thallus are vigorous and with a high concentration, so an optimum seed age for selecting activated seeds is 16 h to 20 h.

3 Determination of Fermentation Medium

A highest viable count of Lactobacillus casei I0B-P9 obtained on the solid medium was used as an index to judge growth activity of the strain.

TABLE 3
Determination of ratio of material to water

| Ratio of Material to Water | Strain Activity (CFU/g) |
|---|---|
| 1:1 | 6.7 × 10⁸ |
| 1:1.25 | 5.3 × 10⁹ |
| 1:1.5 | 8.9 × 10⁹ |
| 1:1.75 | 9.5 × 10⁹ |
| 1:2 | 9.4 × 10⁹ |

An optimum ratio of material to water is 1:1.75, and the highest strain activity is 9.7×10⁹ CFU/g.

On a basis of above experiments, experiments for the inoculation amount were carried out.

TABLE 4
Determination of inoculation amount

| Inoculation Amount (CFU/mL) | Strain Activity (CFU/g) |
|---|---|
| 1 × 10⁶ | 6.3 × 10⁸ |
| 5 × 10⁶ | 4.6 × 10⁹ |
| 1 × 10⁷ | 7.8 × 10⁹ |
| 5 × 10⁷ | 8.9 × 10⁹ |

An optimal inoculation amount is 5 λ10⁷ CFU/mL, and the highest activity was 8.9×10⁹ CFU/g.

On a basis of above experiments, experiments for culture duration in solid state fermentation were carried out.

TABLE 5
Determination of culture duration

| Culture Duration (h) | Strain Activity (CFU/g) |
|---|---|
| 24 | 5.4 × 10⁸ |
| 28 | 6.8 × 10⁸ |
| 32 | 3.1 × 10⁹ |
| 36 | 9.7 × 10⁹ |
| 40 | 9.5 × 10⁹ |
| 44 | 9.3 × 10⁹ |
| 48 | 9.2 × 10⁹ |

Optimal culture duration is 36 h, and the highest viable count was 9.7×10⁹ CFU/g.

To sum up, the optimal culture duration of the agar slant seeds is 24 h, an optimal seed age of activated seeds is 16 to 20 h, the optimum ratio of material to water is 1:1.75, the optimal inoculation amount is 5×10⁷ CFU/mL, and optimal culture duration for solid state fermentation is 36 h.

(1) Experiments were carried out according to an optimal fermentation condition to finally obtain a fermentation sample, and a cultured fermentation product is dried at a high temperature, with a drying temperature of 55±2° C. and drying moisture content being controlled to be less than or equal to 10%, so as to obtain inactivated I0B-P9 fermented postbiotics.

(2) The cultured Lactobacillus casei CGMCC No. 5469 was dried at a high temperature, with a drying temperature of 55±2° C. and drying moisture content being controlled to be less than or equal to 10%, so as to obtain postbiotics of the inactivated Lactobacillus casei. CGMCC No. 5469 is a preservation number in China General Microbiological Culture Collection Center.

### Embodiment 2 Composition Determination of Postbiotics

Composition of postbiotics are measured, in which specific composition and methods are as follows: a measuring method of soybean peptide is that for Soy peptide powder in GB/T22492-2008; a measuring method of soybean isoflavone is a determination method of soybean isoflavone in health food in GB/T 23788-2009, which is high performance liquid chromatography; and a measuring method of free amino acids is a general analytical method for beverage in GB/T 12143-2008.

TABLE 6
degradation of soybean isoflavone

| | | Before | After | Degrada- |
|---|---|---|---|---|
| | Composition | fermentation | fermentation | tion rate |
| Sample name | measured | (mg/100 g) | (mg/100 g) | (%) |
| postbiotics of | genistin | 3.68 ± 0.03 | 1.43 ± 0.04 | 61.14 |
| lactobacillus | glycitin | 16.65 ± 0.04 | 7.63 ± 0.07 | 54.17 |
| casei IOB-P9 | daidzin | 29.54 ± 0.08 | 13.94 ± 0.09 | 52.81 |
| postbiotics of | genistin | 3.35 ± 0.12 | 1.72 ± 0.10 | 51.27 |
| lactobacillus | glycitin | 16.32 ± 0.14 | 9.43 ± 0.15 | 42.21 |
| casei CGMCC | daidzin | 28.46 ± 0.33 | 15.23 ± 0.22 | 46.48 |
| No. 5469 | | | | |

TABLE 7
generation of soybean isoflavone aglycones

| | | Before | After | |
|---|---|---|---|---|
| | Composition | fermentation | fermentation | Fold |
| Sample name | measured | (mg/100 g) | (mg/100 g) | increase |
| postbiotics of | genistein | 2.11 ± 0.03 | 13.32 ± 0.12 | 6.31 |
| lactobacillus | glycitein | 5.43 ± 0.05 | 13.46 ± 0.15 | 2.47 |
| casei IOB-P9 | daidzein | 5.52 ± 0.06 | 25.12 ± 0.21 | 6.70 |
| postbiotics of | genistein | 3.35 ± 0.05 | 8.68 ± 0.17 | 3.57 |
| lactobacillus | glycitein | 5.76 ± 0.14 | 12.59 ± 0.28 | 2.18 |
| casei CGMCC | daidzein | 5.32 ± 0.11 | 17.53 ± 0.14 | 3.29 |
| No. 5469 | | | | |

TABLE 8
Test result of nutrient content

| | Composition | Before | After |
|---|---|---|---|
| Sample name | measured | fermentation | fermentation |
| postbiotics of | free amino | 46.53 ± 0.85 | 119.9 ± 0.94 |
| lactobacillus | acids (mg/100 mL) | | |
| casei IOB-P9 | soybean peptide | 1.32 ± 0.03 | 3.69 ± 0.21 |
| | (g/100 g) | | |
| postbiotics of | free amino | 53.23 ± 0.85 | 97.59 ± 0.75 |
| lactobacillus | acids (mg/100 mL) | | |
| casei CGMCC | soybean peptide | 1.42 ± 0.32 | 2.45 ± 0.11 |
| No. 5469 | (g/100 g) | | |

Biological activity of soybean isoflavones are mainly realized by free aglycones. In postbiotics of I0B-P9, soybean isoflavone glycosides are greatly degraded and soybean isoflavone aglycones are greatly increased, especially genistein and daidzein are increased most significantly, and daidzein also increased. The genistein has certain preventive and therapeutic effects on tumor cells, arterial hypertension, arthritis and proliferative vitreoretinopathy and the like, while the daidzein has certain preventive and therapeutic effects on obesity, while both the genistein and the daidzein have certain preventive and therapeutic effects on diabetes.

Specific bioactive peptides produced in soybean fermentation can serve to regulate action of compounds and show biological activity characteristics, such as antihypertensive, antimicrobial, antioxidant and antidiabetic characteristics.

During a fermentation process, with increasing concentration of thallus, content of produced organic acids gradually accumulates and activates endogenous protease in soybean. In this process, activity of protease and ability to hydrolyze protein of protease are mainly considered. Amino acid nitrogen mainly originates from hydrolysis of protein by the protease, and its content can reflect hydrolysis degree of protein, and the hydrolysis degree of protein directly affects digestibility of soybean protein in vitro.

### Embodiment 3 Ameliorative Effect of Postbiotics of I0B-P9 on Type II Diabetes

1 Experimental Materials

Experimental mice were SPF Kunming male mice, weighing 25=1=2 g.

Feed was high-sugar and high-fat feed (67% of rats and mice basal feed+10% of lard+20% of sucrose+2.5% of cholesterol+0.5% of sodium cholate) and basal feed (feed commercially available).

An experimental group 1 involved postbiotics of inactivated I0B-P9, and a control experimental group 2 involved postbiotics of inactivated Lactobacillus casei CGMCC No. 5469 (of which a preparation method was the same as that of the postbiotics of inactivated I0B-P9).

2 Mouse Modeling and Grouping

A number of SPF Kunming male mice weighing 25±2 g were randomly divided into 4 groups (10 mice in each group): a blank group, a model group, an experimental group 1 (postbiotics of inactivated I0B-P9) and an experimental group 2 (postbiotics of inactivated Lactobacillus casei CGMCC No. 5469). A mouse house was controlled with a temperature of (22±2) ° C., humidity of 50% to 60%, and was circulated day and night for 12 h, in which the mice were fed freely with adaptive feeding with basal feed for 1 week. After 1 week, the model group and the experimental groups were fed with high-fat and high-sugar feed for 3 weeks. Intraperitoneal injection of 10 mg/mL streptozotocin (STZ) at 75 mg per kilogram body weight was carried out from the 4th week, with fasting for about 12 hours before injection and continuous injection for 3 days, so as to establish a diabetic model. At the same time, the blank group was injected with a citric acid buffer solution (0.1 M, pH of 4.5). After modeling, blood samples were taken at tails to randomly determine blood glucose of mice, and if a blood glucose level was larger than 11.1 mmol/L, the modeling was successful. After successful modeling, the mice were intervened with gavage of postbiotics of Lactobacillus casei I0B-P9 and postbiotics of Lactobacillus casei CGMCC No. 5469 at a dose of 75 mg/kg for 4 weeks, and the model group was subjected to gavage of a same amount of normal saline.

3 Data Monitoring

Morphological observation of mice. Water consumption, diet, mental state, hair and urination of mice were observed and recorded.

Determination of fasting blood glucose of mice. After start of experiments, mice were fasted without water deprivation overnight for 12 hours before determination every week, and their fasting blood glucose was measured by tail blood sampling.

Determination of glucose tolerance of mice. In the ninth week of the experiments, the mice were fasted without water deprivation overnight for 12 hours, and 20% of glucose solution was orally administered at a dose of 2 g/kg. Blood samples were collected from the tails to determine blood glucose levels of the mice at the 0th, 15th, 30th, 60th, 90th and 120th min, respectively.

Blood and tissue collection of mice. At the end of the experiments, all of four groups of mice were fasted overnight for 12 hours and then weighed, and blood samples were collected by an eyeball blood collection method and the mice were subjected to neck breaking and sacrificed. Dissection was made and pancreas, liver and kidney were quickly removed and washed in cold physiological saline. The blood samples were centrifuged at 3000×g at 4° C. for 10 min, and serum was collected and stored at −80° C. for later use.

Determination of related indexes for liver. Liver tissue was mixed with 0.1 mmol/L of phosphate buffer solution by a mass ratio of 1:10, homogenized with a homogenizer, centrifuged at 10000×g for 10 min, and supernatant was collected. Content of liver glycogen was determined according to steps in kit instructions.

Determination of serum lipid. Related indexes for serum lipid were determined by a kit.

4 Experimental Results

4.1 Morphology of Mice

By observing activity and mental state of mice, mice in the blank group, model group and experimental groups have normal activity and good mental state. The results show that the mice are in a good mental state after eating the postbiotics of inactivated Lactobacillus casei I0B-P9.

4.2 Blood Glucose of Mice

Referring to FIG. 2, after orally administering of diabetic mice in the experimental group 1 and the experimental group 2, their blood glucose is increased a little at first, and then gradually decreased. Blood glucose reducing effect of the experimental group 1 is better than that of the experimental group 2, for which there is an obvious downward trend compared with the model group, further indicating that it have relieving effect on the blood glucose of mice.

4.3 Glucose Tolerance of Mice

Referring to FIG. 3, after gavage of glucose of the mice, the blood glucose level of the mice reached the highest in about 15 to 30 min. Comparing downward trends of blood glucose in different groups of mice, it is found that the blood glucose level of mice in the blank group reaches the highest at 30 min, and then begins to decrease gradually. The blood glucose levels of mice in experimental group 1 and experimental group 2 decrease rapidly after 30 min, and gradually tend to a level of the blank group. Although the blood glucose level of mice in the model group is in a decreasing trend after 30 min, it was still at a high level, and the blood glucose level was much higher than a normal blood glucose level after 2 hours. Above results show that compared with the model group, glucose tolerance of sick mice is significantly improved in the experimental group 1 and experimental group 2, and effect of the experimental group 1 is superior to that of the experimental group 2.

4.4 Liver Glycogen of Mice

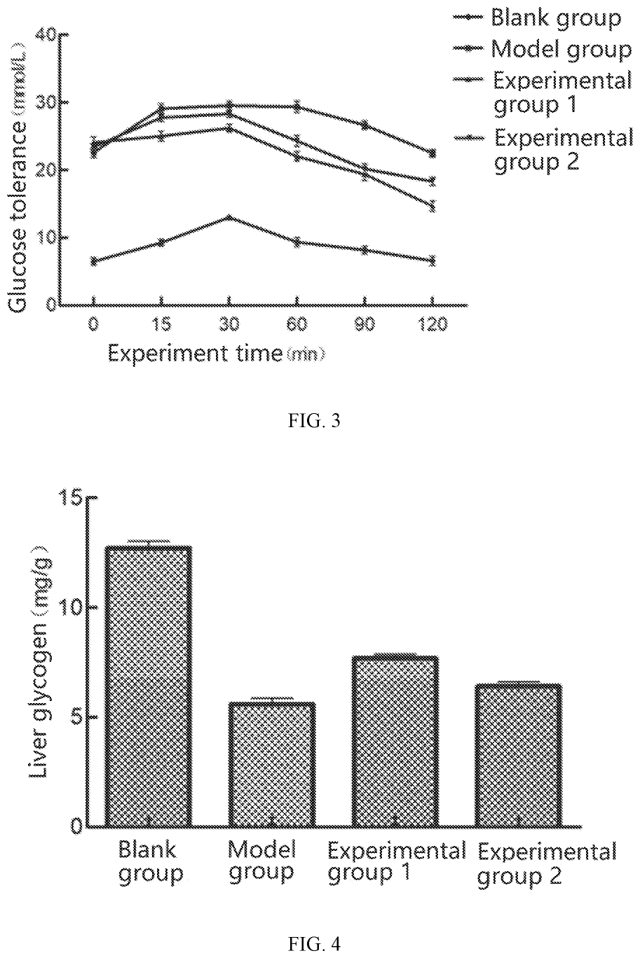

Referring to FIG. 4, after intervention of mice for the experimental group 1 and experimental group 2, content of liver glycogen in diabetic mice increases, but effect of the experimental group 1 is significantly higher than that of the experimental group 2. This may be due to reduction in insulin resistance and enhancement of insulin utilization, and at the same time, with decrease in liver indexes, liver swelling is weakened and liver function is improved, thus increasing the content of liver glycogen.

4.5 Blood Lipid Index of Mice

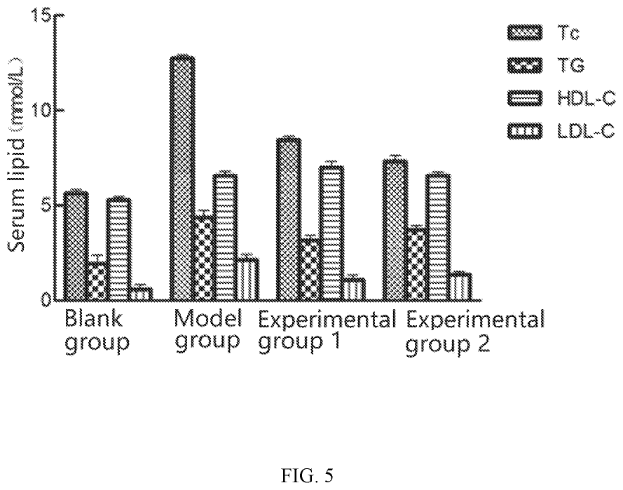

Referring to FIG. 5, diabetic patients generally have severe symptoms of lipid metabolism disorder. Long-term severe lipid metabolism disorder greatly increases probability of complications of coronary heart disease, thus posing great health threat to the patients. As can be seen from FIG. 5, compared with mice in a normal group, total cholesterol (TC), triglyceride (TG) and low-density cholesterol (LDL-C) of mice in the model group is significantly increased. It can be seen from gavage of the mice in the experimental group 1 and the experimental group 2 that content of total cholesterol and triglyceride can be obviously reduced, but reduction amount in the experimental group 1 is superior to that in the experimental group 2. If content of the low-density cholesterol is too high, it is easy to form arteriosclerosis, causing coronary heart disease, stroke and peripheral arterial disease. In the experimental group 1, content of the low-density cholesterol can be reduced. High-density cholesterol is called good cholesterol, which circulates actively in the blood, dissolves and takes away bad cholesterol accumulated at blood vessel walls. In the experimental group 1, content of high-density cholesterol can be increased, while there's no obvious change for the experimental group 2.

To sum up, these experiments studied effects of postbiotics of Lactobacillus casei I0B-P9 and postbiotics of Lactobacillus casei CGMCC No. 5469 on mice with type II diabetes. The results show that the postbiotics of Lactobacillus casei I0B-P9 exhibits good effect on improving dyslipidemia in diabetic mice, and at the same time, it could improve symptoms and related indexes of diabetes, which provides a certain pharmacological basis for adjuvant treatment of diabetes, and provides an idea for developing new products of postbiotics of inactivated Lactobacillus casei I0B-P9.

1. Application of inactivated Lactobacillus casei I0B-P9 fermented postbiotics to preparation of articles for relieving type II diabetes, wherein the Lactobacillus casei I0B-P9 is preserved in China General Microbiological Culture Collection Center, with a preservation number of CGMCC No. 241, and the postbiotics is prepared by: inoculating seed liquid of the Lactobacillus casei I0B-P9 into an activated solid fermentation medium, with the solid fermentation medium being soy powder with a ratio of material to water of 1:1 to 1:2 and with an inoculation amount of 1% to 5% of a dry weight of the solid fermentation medium; and then after being cultured at 37±2° C. for 24 to 48±2 h, dying the cultured fermented product at a high temperature, with a drying temperature of 55±2° C. and drying moisture content being controlled to be less than or equal to 10%, so as to obtain the inactivated I0B-P9 fermented postbiotics.
